(19) United States
(12) Patent Application Publication
(10) Pub. No.: US 2020/0077976 A1
(43) Pub. Date: Mar. 12, 2020

(54) Ultrasonic Diagnostic Device and Volume Data Acquiring Method

(71) Applicant: Hitachi, Ltd., Tokyo (JP)

(72) Inventor: Takanori HIRAI, Tokyo (JP)

(21) Appl. No.: 16/361,673

(22) Filed: Mar. 22, 2019

(30) Foreign Application Priority Data
Sep. 11, 2018 (JP) 2018-170021

(51) Int. Cl. A61B 8/14; A61B 8/00; A61B 8/08

(52) U.S. Cl. CPC A61B 8/14; A61B 8/483; A61B 8/4254

(57) ABSTRACT

A change from a non-contact state to a contact state is determined. Until a change from the contact state to the non-contact state is determined, frame data is sequentially acquired. Volume data is configured with a plurality of pieces of acquired frame data. When the number of pieces of acquired frame data is small, error processing is executed. After determining the acquisition end, the volume data processing is automatically executed.

### BACKGROUND OF THE INVENTION

### 1. Field of the Invention

The present invention relates to an ultrasonic diagnostic device and a volume data acquiring method, and more particularly, to control of acquisition of volume data.

### 2. Description of Related Art

An ultrasound diagnostic device is utilized in the medical field. The ultrasonic diagnostic device is a device for forming an ultrasonic image based on data obtained by transmitting and receiving ultrasonic waves to a living body. The ultrasound diagnostic device is also used in surgical operations. For example, an intraoperative probe abuts against a liver surface exposed after laparotomy, and ultrasonic diagnosis on the liver is performed in this state. An intraoperative probe is disclosed in JP-A-2003-334190.

Recently, an ultrasonic diagnostic device having a real-time virtual sonography (RVS) function is becoming widespread and is expected to be utilized more and more. According to the RVS function, for example, it is possible to simultaneously observe a real-time tomographic image showing a cross-section in a living body and other tomographic images (CT images, MRI images, and the like) showing the same cross-section. Calibration is performed prior to the use of the RVS function. Calibration matches or adjusts a coordinate system of volume data (or frame data) acquired by the ultrasonic diagnostic device with a coordinate system of volume data acquired by another medical device.

The ultrasonic diagnostic device disclosed in WO 2017/038162 has a function of determining whether the ultrasonic probe is in contact with the living body and whether the ultrasonic probe is separated from the living body.

Manual scanning is performed with an ultrasound probe on a body surface for calibration of the coordinate system, volume rendering and other purposes. By the manual scanning, a frame data sequence, that is, volume data is acquired. Here, when a user is requested to instruct acquisition start and acquisition end of the frame data sequence, operational burden or confusion is likely to occur. For example, in a surgical operation, generally, a person who operates the ultrasonic probe and a person who operates the ultrasonic diagnostic device are different from each other. Accordingly, it is not easy to adjust timing between the manual scanning of the ultrasonic probe and the input (for example, the input of the acquisition start or the acquisition end) to the ultrasonic diagnostic device. In the process of performing the manual scanning of the ultrasonic probe, it is desirable that the necessary frame data sequence is naturally acquired without a burden of the user. WO 2017/038162 does not describe a technique for supporting the manual scanning of the ultrasonic probe or the acquisition of the volume data.

### SUMMARY OF THE INVENTION

An object of the present invention is to provide a technique for naturally acquiring a frame data sequence necessary in a process of manual scanning of an ultrasonic probe.

An ultrasonic diagnostic device according to the invention includes: an ultrasonic probe for transmitting and receiving ultrasonic waves; a state identifying unit for identifying a non-contact state where the ultrasonic probe is separated from a living body and a contact state where the ultrasonic probe abuts against the living body based on frame data sequentially obtained by the transmission and reception of the ultrasonic waves; and a control unit for determining acquisition start of a frame data sequence obtained by manual scanning of the ultrasonic probe based on a first state change from the non-contact state to the contact state.

A volume data acquiring method according to the invention includes steps of: identifying a non-contact state where an ultrasonic probe is separated from a living body and a contact state where the ultrasonic probe abuts against the living body based on frame data sequentially obtained by the ultrasonic probe; determining acquisition start of volume data configured with a frame data sequence obtained by manual scanning of the ultrasonic probe based on a first state change from the non-contact state to the contact state; and determining processing start of the volume data while also determining acquisition end of the volume data based on a second state change from the contact state to the non-contact state.

According to the invention, since a necessary frame data sequence is naturally acquired in the process of the manual scanning of the ultrasonic probe, it is possible to reduce the burden on the user.

### BRIEF DESCRIPTION OF THE DRAWINGS

FIG. 1 is a block diagram illustrating an ultrasonic diagnostic device according to an embodiment;

FIG. 2 is a view illustrating an example of a sample point array;

FIG. 3 is a view for describing a calculating method of the sample point array;

FIG. 4 is a flowchart illustrating a processing method of a luminance value sequence;

FIG. 5 is a view for describing standardizing;

FIG. 6 is a view for describing an identifying method of a contact state and a non-contact state;

FIG. 7 is a flowchart illustrating a volume data acquiring method according to the embodiment;

FIG. 8 is a view illustrating an example of an indicator; and

FIG. 9 is a view for describing calibration.

### DESCRIPTION OF EMBODIMENTS

Hereinafter, an embodiment will be described below with reference to the drawings.

### (1) Outline of Embodiment

An ultrasonic diagnostic device according to the embodiment includes an ultrasonic probe, a state identifying unit, and a control unit. The ultrasonic probe transmits and receives ultrasonic waves. The state identifying unit identifies a non-contact state where the ultrasonic probe is separated from a living body and a contact state where the ultrasonic probe abuts against the living body based on frame data sequentially obtained by the transmission and reception of the ultrasonic waves. Based on a first state change from the non-contact state to the contact state, the control unit determines acquisition start of a frame data sequence obtained by manual scanning of the ultrasonic probe.

When acquiring the frame data sequence, a series of operations that includes making the ultrasonic probe abut against a living body surface, scanning the living body surface with the ultrasonic probe while maintaining the abutting state, and detaching the ultrasonic probe from the living body surface, is executed. According to the configuration, in such a series of operations, at least the acquisition start of the frame data sequence is automatically determined, and the frame data sequence is acquired. Since it is unnecessary to instruct the acquisition start, it is possible to reduce a burden on a user. It becomes possible for the user to concentrate on the manual scanning, and it is possible to reduce mistakes in acquiring the frame data sequence. The acquisition start of the frame data sequence means start of managing or storing process of the frame data sequence as a data processing target.

In the embodiment, the control unit further determines acquisition end of the frame data sequence based on a second state change from the contact state to the non-contact state. According to the configuration, since the acquisition end of the frame data sequence is automatically determined, it is unnecessary for the user to instruct the acquisition end. In other words, only by tracing the living body surface with the ultrasonic probe, the acquisition of the necessary frame data sequence is completed. In particular, in a case of performing ultrasonic diagnosis in a surgical operation, the above-described configuration is extremely useful. The acquisition end of the frame data means end of the managing or storing process of the frame data sequence as a data processing target.

The acquisition end may be determined based on the number of pieces of acquired frame data, a scanning distance of the ultrasonic probe, a scanning time of the ultrasonic probe, and the like. When any one among a plurality of ending conditions is satisfied or when all of the plurality of ending conditions are satisfied, the acquisition end may be determined.

In the embodiment, the control unit further determines the start of processing of volume data that serves as a frame data sequence based on the second state change. In the embodiment, the processing of the volume data is calibration for making a coordinate system of the volume data match a coordinate system of other volume data. When using the RVS function, it is necessary to perform the calibration for adjusting the plurality of coordinate systems, and according to the configuration, it is possible to automate coordinate system calibration only by tracing the living body surface with the ultrasonic probe. In the calibration, for example, the position and orientation of a specific part in the volume data are recognized, and a coordinate system is defined with the specific part as a reference. The coordinate system may be defined by other methods. The operation of the ultrasonic diagnostic device may be automatically changed when the acquisition end is determined. For example, a freeze state may be automatically created.

In the embodiment, the control unit executes error processing when the second state change occurs during the manual scanning after determination of the acquisition start. For example, when the number of pieces of acquired frame data is less than a certain value when the acquisition ends, the error processing is executed to encourage to repeat the acquisition or the like. An error may be determined based on a scanning distance or the like.

In the embodiment, the state identifying unit includes a sampling unit for sampling a plurality of luminance value sequences that corresponds to a plurality of depths for each of the frame data, a standardizing unit for standardization of each luminance value sequence, and a determining unit for determining the non-contact state and the contact state based on the plurality of standardized luminance value sequences. In general, a gain or an attenuation amount changes according to the depth. Based on this, the above-described configuration is for performing the state determination from the plurality of luminance values after standardization of each depth.

In the embodiment, the standardizing unit includes a unit for calculating a reference value based on the luminance value sequence for each luminance value sequence, and a unit for standardizing the plurality of luminance values that configures the luminance value sequence for each luminance value sequence by the reference value. As the reference value, for example, an average value, a variance value, or the like may be used.

In the embodiment, the state identifying unit identifies the non-contact state and the contact state based on at least two pieces of frame data arranged spatially. For example, when a constant luminance condition is satisfied over a plurality of frames, the non-contact state or the contact state is identified. According to the configuration, identification accuracy can be enhanced.

In the embodiment, a unit for displaying an indicator for indicating at least one of the non-contact state and the contact state identified by the state identifying unit is provided. By referring to the indicator, it is possible to identify or confirm the state of the ultrasonic probe.

The volume data acquiring method according to the embodiment is executed in the ultrasonic diagnostic device, and particularly, is executed by the control unit or a controller. Specifically, the non-contact state where the ultrasonic probe is separated from the living body and the contact state where the ultrasonic probe abuts against the living body are identified based on the frame data sequentially obtained by the ultrasonic probe. Based on the first state change from the non-contact state to the contact state, the acquisition start of the volume data configured with the frame data sequence obtained by the manual scanning of the ultrasonic probe is determined. Thereafter, based on the second state change from the contact state to the non-contact state, the acquisition end of the volume data is determined, and the start of processing the volume data is determined.

### (2) Details of Embodiment

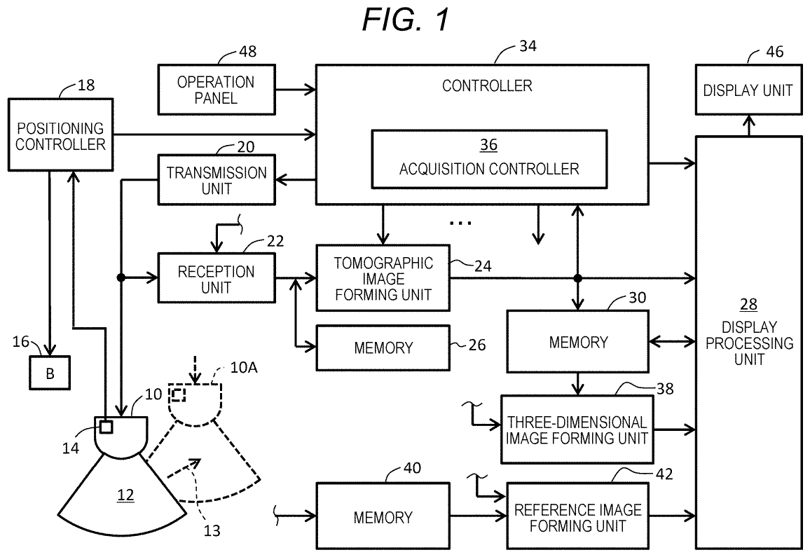

FIG. 1 illustrates an ultrasonic diagnostic device according to the embodiment. The ultrasonic diagnostic device is a device installed at medical institutions, such as hospitals, and forms an ultrasonic image based on received data obtained by transmitting and receiving the ultrasonic waves to and from the living body.

In FIG. 1, an ultrasonic probe 10 is a so-called intraoperative probe used during the surgical operation. In other words, the ultrasonic probe 10 is used while abutting against the surface of an organ exposed in an open state. The organ is, for example, the liver. Other ultrasonic probes may be used.

The ultrasonic probe 10 is configured with a probe head, a cable, and a connector. A vibration element array is provided in the probe head. In the embodiment, the vibration element array is a 1D vibration element array configured with a plurality of vibration elements arranged in an arc shape. The ultrasonic waves are transmitted and received by the vibration element array, and ultrasonic beams are formed at this time. A scanning surface 12 is formed by electronic scanning of the ultrasonic beam. An electronic linear scanning method, an electronic sector scanning method, and the like are known as an electronic scanning method. In the embodiment, an electronic convex scanning method which is one mode of the electronic linear scanning method is adopted. The probe head is held by a plurality of fingers of a surgeon. The 1D vibration element array configured with the plurality of vibration elements which are linearly arranged may be provided in the probe head.

In the embodiment, manual scanning is performed with the probe head along a liver surface while maintaining the state where the transmission and reception surface of the probe head abuts against the liver surface. In FIG. 1, the manual scanning is indicated by a reference numeral 13. The moved probe is indicated by a reference numeral 10A. The manual scanning direction is basically a direction orthogonal to the scanning surface 12. By the manual scanning, a plurality of scanning surfaces 12 is formed at a plurality of spatially different positions, and a plurality of pieces of frame data (a plurality of pieces of received frame data) that corresponds to the plurality of scanning surfaces 12 is acquired. The frame data corresponds to the volume data which will be described later.

A magnetic sensor 14 is provided in the ultrasonic probe 10, specifically, in the probe head. A magnetic field (three-dimensional magnetic field) for positioning is generated by a magnetic field generator 16, and the magnetic field is detected by the magnetic sensor 14. A detection signal output from the magnetic sensor 14 is sent to a positioning controller 18. A driving signal is sent from the positioning controller 18 to the magnetic field generator. Based on the detection signal output from the magnetic sensor 14, the positioning controller 18 calculates the position and posture of the probe head where the magnetic sensor 14 is provided, that is, the position and posture of the scanning surface 12. In other words, in the embodiment, positional information is calculated for each frame data. The calculated positional information is output to a controller 34. The positioning controller 18 may be configured as an electronic circuit. The positioning controller 18 may be integrated in the controller 34. The magnetic sensor 14, the magnetic field generator 16, and the positioning controller 18 configure a positioning system.

A transmission unit 20 is a transmission beamformer that supplies a plurality of transmission signals in parallel to a plurality of vibration elements that configure the vibration element array in the transmission, and is configured as an electronic circuit. A reception unit 22 is a reception beamformer that performs phase adjustment addition (delay addition) of a plurality of received signals output in parallel from the plurality of vibration elements that configure the vibration element array in the reception, and is configured as an electronic circuit. The reception unit 22 includes a plurality of A/D converters, a detection circuit, and the like. Beam data is generated by the phase adjustment addition of the plurality of received signals by the reception unit 22. Each piece of frame data is configured with a plurality of pieces of beam data arranged in an electronic scanning direction. Each beam data is configured with a plurality of pieces of echo data arranged in a depth direction. At a rear end of the reception unit 22, a beam data processing unit is provided, but is not illustrated in the drawing.

In the embodiment, a tomographic image forming unit 24 is configured with a processor or the like including a digital scan converter (DSC). The DSC has a coordinate conversion function, a pixel interpolation function, a frame rate conversion function, and the like. The tomographic image forming unit 24 sequentially forms a plurality of tomographic images based on a plurality of sequentially input frame data. The identity of each tomographic image is the frame data (display frame data) after the coordinate conversion. The frame data is configured with a plurality of pieces of luminance data (luminance values) aligned in x direction and y direction. The volume data is configured with the spatially arranged volume data. The plurality of pieces of frame data sequentially output from the tomographic image forming unit 24 is sent to a display processing unit 28 and the controller 34 in the configuration example illustrated in FIG. 1.

The controller 34 functions as a control unit, and in the embodiment, is configured with a CPU and an operation program. The controller 34 controls the operation of each configuration illustrated in FIG. 1, and in the embodiment, has a volume data acquisition control function. The function is illustrated as an acquisition controller 36 in FIG. 1. The acquisition controller 36 functions as a state identifying unit and an acquisition control unit and further functions as a sampling unit, a standardizing unit, and a determining unit. More specifically, in the process of the manual scanning of the ultrasonic probe 10, the acquisition controller 36 identifies the abutting (contact) state of the ultrasonic probe against the organ surface and the detached (non-contact) state of the ultrasonic probe from the organ surface based on the plurality of pieces of frame data sequentially output from the tomographic image forming unit 24, and automatically determines the acquisition start and the acquisition end of the frame data sequence which is the processing target based on the identification result.

In a memory 30, under the control of the controller 34, a specific frame data sequence, that is, the volume data is stored. Specifically, in the series of frame data output from the tomographic image forming unit 24, a plurality of pieces of frame data generated within the period from the time when the acquisition controller 36 determines the acquisition start to the time when the acquisition controller 36 determines the acquisition end, is stored in the memory 30. The frame data configures the volume data that serves as a processing target or a processing unit. The memory 30 includes a three-dimensional storage space and corresponds to a 3D memory. When storing each frame data in the memory 30, the positional information may be stored in association with each frame data. In other words, the positional information may be acquired within the period from the acquisition start to the acquisition end. A plurality of pieces of stored positional information indicates a spatial relationship between a plurality of pieces of frame data. The positional information is used for image processing and is used for coordinate system calibration. In the memory 30, each frame data may be written in a storage region specified from each positional information.

A three-dimensional image forming unit 38 forms a three-dimensional image based on the volume data stored in the memory 30. The three-dimensional image is an image stereoscopically illustrating the tissue. When forming the three-dimensional image, a volume rendering method, a surface rendering method, or the like is used.

A memory 40 stores volume data acquired by another medical device (for example, an X-ray CT device, an MRI device, or other ultrasonic diagnostic devices) therein. In the embodiment, the volume data is acquired from the same object, specifically, the volume data is acquired from a three-dimensional region including the liver in the same person. Based on the volume data in the memory 40, a reference image forming unit 42 forms a tomographic image, a three-dimensional image, or the like as a reference image. When executing the RVS function, the reference image forming unit 42 forms a tomographic image showing the same cross-section as the currently displayed tomographic image. Each of the three-dimensional image forming unit 38, the reference image forming unit 42, and the display processing unit 28 is configured with, for example, a processor. The units may be configured with a single processor. The units may be realized as functions of the controller 34.

In the embodiment, the controller 34 has a coordinate system calibration function. When executing the function, the volume data stored in the memory 30 is read by the controller 34. The controller 34 automatically recognizes a predetermined object (image of a predetermined part) in the volume data, and defines the coordinate system based on the predetermined object. The predetermined object is, for example, a portal vein image that corresponds to a portal vein in the liver. A coordinate system defined with the object as a reference is applied to the volume data. The coordinate system is also defined for the volume data stored in the memory 40 by the same method described above. Otherwise, the volume data in which the coordinate system has already been defined is stored in the memory 40. By the above-described process, it becomes possible to match the coordinate system between the two pieces of volume data. When recognizing the object, for example, a machine learning type identifier may be used. The identifier is configured with, for example, a convolutional neural network (CNN).

The tomographic image data, the three-dimensional image data, the reference image data and the like are input to the display processing unit 28. The display processing unit 28 has an image synthesis function, a color processing function, a graphic image generation function, and the like. An indicator which will be described later is generated by the graphic image generation function. A display image to be displayed on the display unit 46 is formed by the display processing unit 28. The display unit 46 is configured with an LCD, an organic EL display device, or the like. An operation panel 48 connected to the controller 34 is an input device, and includes a plurality of switches, a plurality of buttons, a track ball, a keyboard, and the like.

In the embodiment, the frame data sequence after the coordinate conversion is acquired, but the frame data sequence before the coordinate conversion may be acquired. In this case, for example, the frame data sequence before the coordinate conversion is stored in a memory 26. When using the frame data sequence in the memory 26, after the frame data sequence read from the memory 26 is processed in the tomographic image forming unit 24 in the same manner as described above, the processed frame data sequence is transferred to the controller 34 or the like.

As described above, the acquisition controller 36 has the function of identifying the contact state and the non-contact state, and by using the function, the acquisition controller 36 determines the first state change from the non-contact state to the contact state and determines the second state change from the contact state to the non-contact state in the process of the manual scanning. The acquisition controller 36 determines the acquisition start when the first state change occurs and determines the acquisition end when the second state change occurs. Hereinafter, the processing and control by the acquisition controller 36 will be described in detail below.

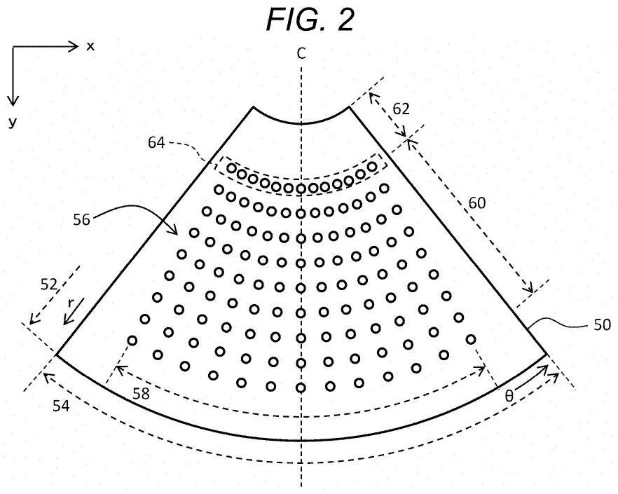

In FIG. 2, frame data 50 is illustrated. The frame data 50 is frame data after the coordinate conversion generated at a certain time. An xy coordinate system is a display coordinate system. An rθ coordinate system is a transmission and reception coordinate system. r indicates the depth direction, and θ indicates the electronic scanning direction. A depth range 52 of the frame data 50 corresponds to a set diagnosis range. A range 54 in a θ direction of the frame data 50 corresponds to the electronic scanning range.

In the embodiment, for each frame data 50, a sample point array that spreads in the entire frame data 50 is set, and the luminance value is referred to at each sample point. Accordingly, a luminance value array 56 is acquired. Each of the luminance values corresponds to echo values. The luminance value array 56 includes a plurality of pixel value sequences that corresponds to a plurality of depths, and each pixel value sequence is configured with a plurality of luminance values arranged in the θ direction. For example, the pixel value sequence 64 is configured with dozens of luminance values arranged in an arc shape.

When designating the sample point array, for example, a start depth and an end depth are specified. A range 60 therebetween is the width of the sample point array in the depth direction. Similarly, a range 58 is designated in the θ direction, and the range 58 is a width of the sample point array. Incidentally, the sample point array does not extend to a probe proximity range 62, but rather, the sample point array is set on the side deeper than the probe proximity range 62. In the probe proximity range 62, striped patterns are likely to be generated due to multiple reflections, and reference ranges are set so as not to refer to the striped patterns.

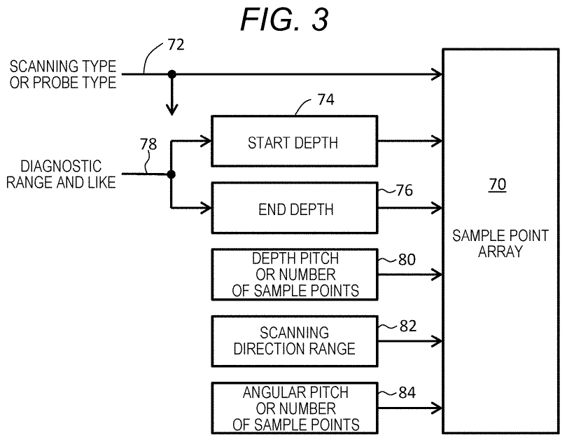

For example, as illustrated in FIG. 3, a sample point array 70 includes a scanning type or a probe type 72, a starting depth 74, an ending depth 76, a number of sample points 80 in a depth pitch or a depth direction, an electronic scanning direction range 82, a number of sample points 84 in an angular pitch or the electronic scanning direction, and the like. In this case, the start depth 74 and the end depth 76 may be automatically set based on a diagnostic range 78 and the like. Information other than the information illustrated in FIG. 3 (for example, a target organ, a medical examination subject) may be referred to, and the sample point array 70 may be determined based on the preset information. Generally, the same sample point array 70 is applied to each frame data. When a rectangular scanning surface is formed, a rectangular sample point array is formed. In other words, an external shape of the sample point array is determined according to the mode of the scanning surface.

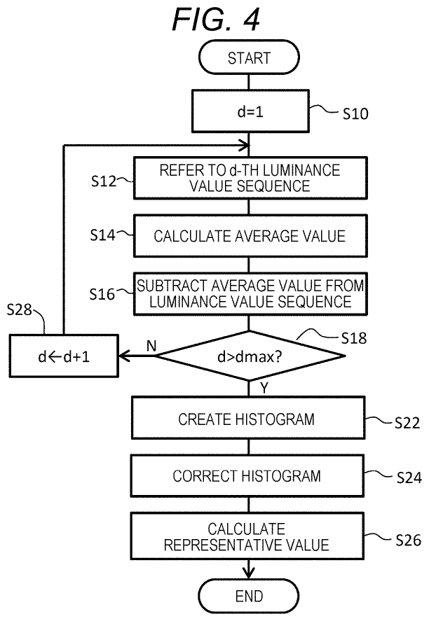

In FIG. 4, a method of calculating a representative value is illustrated as a flowchart. In the embodiment, for each acquired frame data, a representative value representing the frame data is calculated, and based on the representative value, a contact state or a non-contact state is identified. Hereinafter, this will be described in detail.

In S10, a depth parameter d is initialized, and in the illustrated example, 1 is assigned to d. By the d, a specific luminance value sequence is designated from the plurality of luminance value sequences arranged in the depth direction. In S12, the d-th luminance value sequence is referred to. In S14, a reference value for standardization is calculated based on the luminance value sequence. In the embodiment, the reference value is an average value. In S16, the average value is subtracted from each luminance value that configures the luminance value sequence. If a negative value is generated, the value is replaced with 0 or invalidated. In the frame data, a gain or an attenuation amount generally changes along the depth direction. By subtracting or standardizing the above-described average value, it becomes possible to eliminate or reduce influence of change in the gain or the attenuation amount along the depth direction.

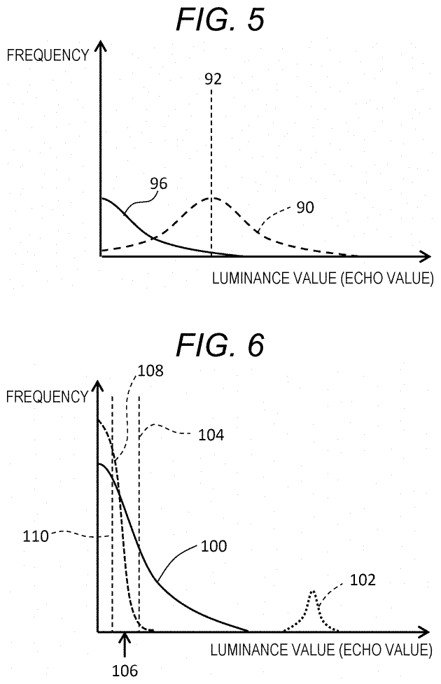

A specific example of the processing in S16 described above will be described with reference to FIG. 5. In FIG. 5, a histogram 90 that corresponds to one luminance value sequence is illustrated. The histogram 90 is for description and was not created by S16 described above. The horizontal axis indicates the luminance value, and the vertical axis indicates the frequency (the number of pixels). Due to the subtraction of an average value 92, the histogram 90 changes to a histogram 96. Even when the center position of the histogram changes according to the depth, it becomes possible to align the center positions of the histogram after the standardization. Accordingly, each of the luminance value sequences may be standardized by other methods. For example, standardization may be performed using a median value, standard deviation, or the like. The standardization may be performed by using the total average value over the entire frame.

Returning to FIG. 4, in S18, it is determined whether d exceeds a maximum value dmax. When d does not exceed dmax, d is incremented by 1 in S28, and each step after S12 is repeatedly executed. Accordingly, a plurality of luminance value sequences arranged in the depth direction are sequentially standardized. When it is determined that d exceeds dmax by S18, in S22, a histogram is created based on a plurality of standardized luminance value sequences. In S24, the created histogram is corrected. Specifically, in the histogram, the number of pixels is sequentially referred to from the luminance value 0 to the high luminance side, and after the number of pixels is detected as 0, all the number of pixels thereafter are replaced with 0. In other words, an isolated part on the high luminance side is removed. In S26, a representative value is calculated based on the corrected histogram. In the embodiment, the representative value is an average value (overall average value). By comparing the representative value with a threshold value, the contact state or the non-contact state is identified.

The processing in S22 to S26 will be specifically described with reference to FIG. 6. A histogram 100 is a standardized histogram that corresponds to the contact state. A histogram 108 is a standardized histogram that corresponds to the non-contact state. For example, when an isolated part 102 is generated, the isolated part 102 is removed in S24. Accordingly, it becomes possible to determine the contact state and the non-contact state without being affected by specific high luminance noise. An average value 104 of the histogram 100 is equal to or greater than the threshold value, and in this case, the contact state is determined. Meanwhile, an average value 110 of the histogram 108 is less than the threshold value, and in this case, the non-contact state is determined.

According to the method illustrated in FIG. 4, since the luminance value is sampled from substantially the entire frame data, it is possible to perform the state determination without being affected by local luminance value distribution. It is advantageous in that since a probe proximity region is excluded from the sampling target, it is hardly affected by multiple reflections, and since standardization is performed, it is hardly affected by depth dependency. By correcting the histogram, even when high-intensity noise (for example, reflected wave from a bone) is generated, it is also advantageous that it is hardly affected by the high-intensity noise.

In the description above, although the contact state is determined when the representative value exceeds the threshold value, the contact state may be determined when the representative value continuously exceeds the threshold value over the plurality of frames. Similarly, in the description above, although the non-contact state is determined when the representative value is less than the threshold value, the non-contact state may be determined when the representative value is continuously less than the threshold value over the plurality of frames.

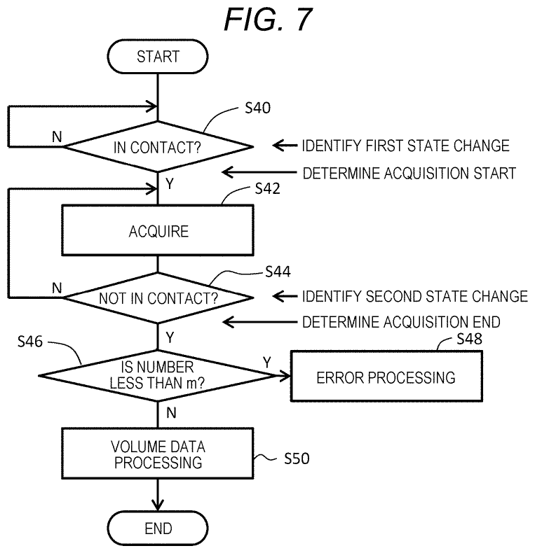

FIG. 7 illustrates a volume data acquiring method according to the embodiment. First, a flow of an operation of the ultrasonic probe to be performed by the user (surgeon) will be described. The ultrasonic probe is held by the user when the electronic scanning of the ultrasonic beam is repeatedly executed. Subsequently, the ultrasonic probe abuts against the scanning start position on the liver surface, and then the ultrasonic probe is moved in a parallel movement mode along the liver surface while maintaining the contact state, and the ultrasonic probe is separated from the liver surface at a standard position after a certain scanning distance has passed or when the scanning is completed. In the process of such a series of operations, each of the following steps are automatically executed.

In S40, the contact state is identified by the method illustrated in FIGS. 4 to 6, that is, a change (first state change) from the non-contact state to the contact state is identified. In other words, in S40, when the ultrasonic probe comes into contact with the liver surface, the liver surface is specified. At this time, it is unnecessary for the user to give an instruction for the acquisition start. S42 is a step of acquiring the frame data. In S44, the non-contact state is identified by the method illustrated in FIGS. 4 to 6, that is, a change (second state change) from the contact state to the non-contact state is identified. As long as the contact state continues, that is, while the manual scanning of the ultrasonic probe continues, the step of S42 is repeatedly executed, and the plurality of pieces of frame data generated at the display frame rate are sequentially acquired.

In S44, the acquisition end is determined based on the identification of the second state change. In other words, the acquisition end is automatically determined when the ultrasonic probe is separated from the liver surface. By the determination of the acquisition end, the storage of the frame data is completed. At this time, it is unnecessary for the user to give an instruction for the acquisition end.

In S46, it is determined whether the number of pieces of stored frame data is less than m, and when the number of pieces of frame data is less than m, the error processing is executed in S48. For example, an image that encourages to repeat the manual scanning is displayed on the display screen. By such error processing, when the necessary volume data cannot be obtained, it becomes possible to notify the situation to the user. As a result, it becomes possible to ensure the appropriateness of the subsequent volume data processing. m is 20 or 30, for example.

In S46, when it is determined that m or more pieces of frame data has been acquired, the volume data processing is executed in S50. Even here, it is unnecessary for the user to instruct the start of the processing. In the embodiment, the volume data processing in S50 is coordinate system calibration. In S50, three-dimensional image processing based on the volume data may be executed.

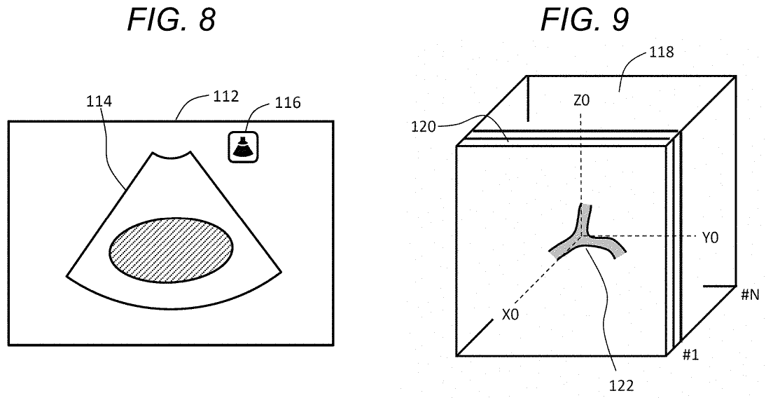

FIG. 8 illustrates a display example. A real time tomographic image 114 is displayed in a display screen 112, and an indicator 116 is displayed in the vicinity thereof. The indicator indicates the contact state and continues to be displayed in the contact state. In other words, during the manual scanning period, the indicator 116 is displayed together with the real time tomographic image 114. Accordingly, it becomes possible for the user to confirm that the contact state is formed correctly. The indicator indicating the non-contact state may be displayed.

FIG. 9 illustrates the coordinate system calibration. By the manual scanning of the ultrasonic probe, frame data 120 from #1 to # N which are spatially arranged are acquired. The position of each frame data 120, more specifically, the position of each echo data that configures each frame data is specified by the positional information obtained by the positioning system. A plurality of pieces of frame data 120 configures volume data 118. The volume data 118 is analyzed and an object 122 included therein is identified. The object 122 is, for example, a portal vein image that corresponds to a portal vein in the liver. An XYZ coordinate system is defined in the volume data 118 with reference to the object 122. In FIG. 9, the reference coordinates in the object 122 are indicated by (X0, Y0, Z0). The same processing as described above may be applied to the volume data acquired by another medical device, and the coordinate systems may be made to match each other between the two pieces of volume data. The coordinate system of the volume data acquired by the ultrasonic diagnostic device may be adapted to the volume data of which the coordinate system has already defined.

According to the above-described embodiment, simply by tracing the organ surface or the living body surface with the ultrasonic probe by the user, necessary volume data is automatically acquired. Here, the operation of the operation panel of the ultrasonic diagnostic device is unnecessary, and it is unnecessary for the user to be conscious of the acquisition itself of the frame data. Moreover, according to the above-described embodiment, since the coordinate system calibration is automatically executed after the manual scanning, it is unnecessary for the user to be conscious of the acquisition of the frame data.

In the above-described embodiment, the acquisition end is determined based on the frame data, but the acquisition end may be determined based on the number of pieces of frame data, the scanning distance, the scanning time, and the like. In this case, the user may be notified of satisfaction of the acquisition end condition. When any one of the plurality of end determination conditions is satisfied or when all of the plurality of end determination conditions are satisfied, the acquisition end may be determined.

What is claimed is:

1. An ultrasonic diagnostic device comprising:
an ultrasonic probe for transmitting and receiving ultrasonic waves;
a state identifying unit for identifying a non-contact state where the ultrasonic probe is separated from a living body and a contact state where the ultrasonic probe abuts against the living body based on frame data sequentially obtained by the transmission and reception of the ultrasonic waves; and
a control unit for determining acquisition start of a frame data sequence obtained by manual scanning of the ultrasonic probe based on a first state change from the non-contact state to the contact state.

2. The ultrasonic diagnostic device according to claim 1, wherein
the control unit further determines acquisition end of the frame data sequence based on a second state change from the contact state to the non-contact state.

3. The ultrasonic diagnostic device according to claim 2, wherein
the control unit further determines start of processing volume data that is the frame data sequence based on the second state change.

4. The ultrasonic diagnostic device according to claim 3, wherein
the processing of the volume data is calibration for making the coordinate system of the volume data match the coordinate system of other volume data.

5. The ultrasonic diagnostic device according to claim 2, wherein
the control unit executes error processing when the second state change occurs in the middle of the manual scanning after determination of the acquisition start.

6. The ultrasonic diagnostic device according to claim 1, wherein
the state identifying unit includes
a sampling unit for sampling a plurality of luminance value sequences that corresponds to a plurality of depths for each of the frame data,
a standardizing unit for performing standardization for each luminance value sequence, and
a determining unit for determining the non-contact state and the contact state based on the plurality of standardized luminance value sequences.

7. The ultrasonic diagnostic device according to claim 6, wherein
the standardizing unit includes
a unit for calculating a reference value based on the luminance value sequence for each luminance value sequence, and
a unit for standardizing the plurality of luminance values that configures the luminance value sequence for each luminance value sequence by the reference value.

8. The ultrasonic diagnostic device according to claim 1, wherein
the state identifying unit identifies the non-contact state and the contact state based on at least two pieces of frame data arranged spatially.

9. The ultrasonic diagnostic device according to claim 1, further comprising:
a unit for displaying an indicator indicating at least one of the non-contact state and the contact state identified by the state identifying unit.

10. A volume data acquiring method comprising steps of:
identifying a non-contact state where an ultrasonic probe is separated from a living body and a contact state where the ultrasonic probe abuts against the living body based on frame data sequentially obtained by the ultrasonic probe;
determining acquisition start of volume data configured with a frame data sequence obtained by manual scanning of the ultrasonic probe based on a first state change from the non-contact state to the contact state; and
determining processing start of the volume data while also determining acquisition end of the volume data based on a second state change from the contact state to the non-contact state.
